(19) United States
(12) Patent Application Publication
(10) Pub. No.: US 2010/0235180 A1
(43) Pub. Date: Sep. 16, 2010

(54) Synergistic Medicodental Outpatient Imaging Center

(76) Inventor: William Atkinson, Orlando, FL (US)

Correspondence Address: Dr. William Atkinson, 2881 Telstar Avenue, Orlando, FL, 32805, US

(21) Appl. No.: 12/401,617

(22) Filed: Mar. 11, 2009

(51) Int. Cl. G06Q 50/00; G01D 18/00; G06Q 40/00; A61C 8/00; A61B 5/055

(52) U.S. Cl. 705/2; 378/207; 705/4; 705/3; 433/201.1; 600/411

(57) ABSTRACT

The invention discloses a method and configuration for a medical and dental outpatient diagnostic imaging center. The medical and dental diagnostic imaging center will house a plurality of medial and dental modalities within one center. Also, onsite is a CAD/CAM milling unit and the accessory hardware equipment, and the technology used for creating a rapid prototype medical and dental modeling The present invention also includes other technologies and equipment to improve the productivity of the medial and dental outpatient diagnostic imaging center. The unique equipment configuration and the machine sequence pattern offers many benefits to healthcare delivery in that it provides a service for both the fields of medicine and dentistry.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates to a method and configuration for a medical and dental imaging center.

2. Description of Related Art

As a field of scientific endeavor, depending on the context, medical imaging constitutes a sub-discipline of biomedical engineering, medical physics, or medicine. Research into the application and interpretation of medical images is usually the preserve of radiology and the medical sub-discipline relevant to medical condition or area of medical science (neuroscience, cardiology, psychiatry, psychology, etc) under investigation.

Conventionally, radiology includes diagnostic imaging, interventional radiology, and radiation oncology. Imaging procedures use energy waves to penetrate human tissue and generate images of the body, which can be recorded on film or digitized for display on a video monitor. Diagnostic imaging procedures are used to diagnose diseases and physical injuries and are performed in hospitals, physician's offices, outpatient imaging centers and mobile imaging centers.

In essence, medical imaging refers to the techniques and processes used for medical science, including the study of normal anatomy and physiology. Conventionally, medical imaging facilities and practices have involved developing and using technologies and modalities that have been utilized by physicians and sub-specialists within medicine as a starting point for the establishment and/or confirmation of an initial diagnosis. Once an image of the pertinent autonomy is generated, it is useful tool for examining, diagnosing, or reaching a diagnostic conclusion.

Today, diagnostic facilities and outpatient centers are extremely limited in that they cater to referring physicians and do not facilitate diagnostic scans and interpretation for the dental healthcare providers within the same facility. Thus, there has been a continuing need for improvement of the facilities because many diagnostic imaging facilities are one dimensional in that they only focus on one field while many injuries and pathologies may sometimes encompass medical and dental diagnostic work.

### BRIEF SUMMARY OF THE INVENTION

Considered broadly, the present inventions method of healthcare delivery will greatly enhance the field of radiography by offering a new configuration for operating a plurality of medial and dental modalities within one center. The invention relates directly to specific equipment configurations and machine sequence patterns within a imaging center, for both the fields of dentistry and medicine.

A principal object of the present invention is to create a diagnostic center that is configured to handle a wide variety of diagnostic processes and technologies focused toward the fields of medicine, dentistry, and dental laboratory. By configuring the selected equipment according to the present invention the outpatient imaging center will provide a benefit by enhancing productivity of the outpatient imaging center to physicians, dentists, referring third party dental labs, and their clients.

Another embodiment of the present imaging center invention is a plurality of medical and dental imaging modalities in one location. Moreover, said imaging center can be located in any of the following locations, including: a centralized outpatient service center, a public funded health facility, a community clinic, a mobile health center, an automobiles, vans, trucks, aircraft, boats or any variation of marine vessel capable of housing said medical and dental imaging modality.

In accordance with the objective of the present invention, the preferred embodiment of the imaging center will comprise the following medical and dental components:

Multi-Slice Computed Tomography (CT) component: a CT utilizes a computer to direct the movement of an x-ray tube to produce multiple cross sectional images of a particular organ or area of the body. The CT is used to detect tumors and other conditions affecting bones and internal organs and used to detect the occurrence of strokes, hemorrhages, and infections. The CT provides higher resolution images than conventional x-rays but soft tissue swelling and edema are not as well defined as those produced by magnetic resonance.

Magnetic Resonance Imaging (MRI) component: MRI utilizes a strong magnetic field in conjunction with low energy electromagnetic waves which are processed by a computer to produce high-resolution images of body tissue including the brain, spine, abdomen, heart and extremities. Unlike CT and conventional x-rays, MRI does not utilize ionizing radiation, which can cause tissue damage in high doses.

Mammography component: Mammography is a specialized form of radiology utilizing low dosage x rays to visualize breast tissue and is the primary screening tool for breast cancer. Mammography procedures also include the biopsy of cells to assist in the diagnosis of breast cancer. New digital mammography techniques are even more sensitive in detecting abnormalities of breast tissue.

Ultrasound component: Ultrasound Imaging utilizes high-frequency sound waves to develop images of internal organs, unborn fetuses and the vascular system. Ultrasound has widespread applications, including procedures in obstetrics gynecology and cardiology.

Cone Beam CT Scanning component: A type and variation of three dimensional computed tomography (CT) in which the source(usually of x-rays)describes a helical trajectory relative to the object while a two dimensional array of detectors measures the transmitted radiation on part of a cone of rays emanating from the source. In practical helical cone beam X-ray CT machines, the source and array of detectors are mounted on a rotating gantry while the patient is moved axially at a uniform rate. Previous and current multi-slice CT scanners imaged one slice at a t time by rotating source and one dimensional array of detectors while the patient remained static. The helical scan reduces the X-ray dose to the patient by one fourth of what a conventional multi-slice CT would require for a given resolution while scanning more quickly. This is however at the cost of greater mathematical complexity and time in the reconstruction of the image as compared to a conventional multi-slice CT which would require less reconstruction time during the reconstruction phase once all data points are collected from the initial scan

CAD/CAM Dental Milling Component: This component would proceed in the typical sequence; the dentist would create an impression of the site to be restored. The impression is sent to a lab, the impression is poured into a model, called a die. Typically this initial lab will send this die to a third party CAD/CAM milling dental lab. Because many commercial labs do not specialize in the CAD/CAM fabrication of substructures in preparation for final restorations, another third party CAD/CAM milling dental lab will needed once the die complete. Once the third party CAD/CAM milling dental lab receives the die they utilize a CAD/CAM scanner with hardware that picks up all points of information. Subsequently, once all points of information are collected, a lab technician is needed to utilize computer-aided design software and will design a 3D model of the substructure or framework in preparation for the final restoration that will be completed by the referring dental laboratory. Eventually, after the CAD/CAM milling technician has gone through the design process utilizing the design software the next step is the milling process which employs the digitized 3D model and CAD/CAM hardware milling unit technology to mill the restoration, and then send it back to the original referring dental laboratory. Another objective of the preferred embodiment is to have the component needed for the CAD/CAM milling technology onsite in the outpatient imagine center. By doing this the invention will provide a savings of time and cost, amongst other things to a healthcare imaging facility.

Static and Dynamic Sialography/Rapid Prototype Medical modeling component: This component is the automatic construction of physical objects using solid freeform fabrication. The first technique for rapid prototyping became available in the late 1980's and is used to produce models and prototype parts. And have most recently been utilized within the field of Medicine and Dentistry in the fabrication of pre-surgical preparation models as well for stent fabrication. The process of rapid prototype medical modeling works by taking a virtual design from a computer aided design (CAD) or animation modeling software, transforms them into thin virtual, horizontal cross-sections and then creates each cross section in physical space, one after the next until the model is fabricated. With additive fabrication, the machine reads in data from a CAD drawing and lays down successive layers of liquid, powder, or sheet material, and in this way builds up the model from a series of cross sections. These layers, which correspond to the virtual cross section from the CAD model, are joined together or fused automatically to create the final shape. The primary advantage to additive fabrication is its ability to create almost any shape or geometric feature. The standard data interface between CAD software and the machines is the STL file format. The STL file format is utilized to create a continuous surface of the preoperative model, stent, or implant fabrication. STL file approximates the shape of a part or assembly using triangular facets. Smaller facets produce a higher quality surface. The word “rapid” is relative: construction of a model with contemporary methods can take approximately several hours to several days. In further accordance with the purpose of the present invention this component would enhance productivity by completing the fabrication in the outpatient imaging center.

Positron emission tomography (PET): Positron emission tomography is primarily used to detect diseases of the brain and heart. Similar to nuclear medicine, an isotope is incorporated into a substance used by the body such as glucose which is absorbed by the tumor of interest. PET scans are often viewed in conjunction to traditional multi-slice (CT) scans, which can be performed on the same equipment without moving the patient. This facilitates the tumors detected by the PET scan be viewed next to the rest of the patients anatomy detected by the CT scan.

In accord with the preferred embodiment, the outpatient imaging center will comprise a plurality of medical and dental modalities within one center. Accordingly, the center will comprise of a licensed physician, dentist, or a staff radiologist who specializes in the sub-specialty of radiology and Oral maxillofacial radiology. In addition, the imaging center will have certified dental lab technologist that is certified in handling the CAD/CAM milling technology.

In yet further accordance with a preferred embodiment, the imaging center will utilize an existing technology that is capable of handling, storing, printing, and transmitting information in medical imaging standard language. The purpose of the technology is to serve as a common communications language among various hardware and software components. The communication protocol is an application protocol that uses TCP/IP to communicate between systems. The vast majority of medical imaging facilities utilize (DICOM) or Digital Imaging and Communication or comparable alternatives to serve for transmitting information in the imaging standard language. DICOM files can be exchanged between two entities that are capable of receiving image and patient data in DICOM format. DICOM includes a file format definition and a network communication protocol. Further DICOM enables the integration of scanners, servers, workstations, printers, and network hardware from multiple manufactures into a picture archiving and communication system (PACS) or a comparable technology.

Still further in accordance with present invention, the imaging center's staff radiologist will serve as the primary conduit for the interpretation of the radiographic scans. The staff radiologist will interpret scans of the relevant part of the patient's anatomy and distribute all of the preliminary and final reports to referring physicians. In addition to working within our facility the staff radiologist within our process will also work offsite performing radiological analysis or “teleradiology” services for other facilities.

In yet further accordance with a preferred embodiment of the present invention, the outpatient imaging center will maintain some form of a practice management software in addition to other information technology management software or a Radiology Information System package to create patient flow efficiencies. Generally, the said software serves as a viable means for retrieving a patient's insurance information, as well as treatment records. Some imaging facilities presently use a Radiology Information System (RIS) component along with a Hospital Information System (HIS) to monitor the work flow of radiology of the center, all the way from the patient check in, to scheduling, billing, shipping and order fulfillment records. The said software is especially ideal for generating medical records and management reporting for the CAD/CAM milling dental crown and bridge substructures along with customized implant abutments, customized implant abutments.

A still further object of the invention is to include in the said imaging center several mechanisms for enhancing productivity. One such mechanism is the incorporation of a electronic LCD touch screen unit, Kiosk, or a comparable technology for managing patient flow. The LCD unit is a more efficient method for handling patient check-in then conventional methods. The LCD unit operates like a touch screen monitor. Accordingly, the said unit will provide instant real time third party insurance verification for patients of the practice. The said unit can be mounted to the wall or it can stand alone like an airport check-in kiosk. Upon arrival to the center the patient will input their identifying information to check-in. In some instances, the patient can swipe their insurance membership card, assuming it contains a magnetic strip or comparable technology. In that case a magnetic strip reader will be located in immediate proximity to the LCD touch screen.

Moreover, in accordance with the present invention a preferred embodiment of the imaging center will incorporate a voice recognition system into the staff radiologist workstation. The Voice Recognition system or some other automated dictation system is ideal when linked to a RIS system or the picture archiving communication system for the advantage of preparing preliminary and final reports where manual transcription is not necessary.

In accordance with the preferred embodiment of the imaging center, still further a method for enhancing patient flow efficiencies between the front desk personnel and clinical personnel. One means for improving said patient flow efficiencies is to have all staff members, employees, and other personnel utilize an interoffice communication system in the office, such as a two way radio and its derivatives or a comparable intercom technology.

Moreover, the outpatient imaging center will utilize an uninterruptible back up power systems. One such device capable of this is a power supply termed “UPS” or Uninterruptible Power Supply, isolation transformers and generators to provide uninterrupted, conditioned, clean power to the critical loads.

A still further object of the invention, the outpatient imaging center is also to provide service to utilize both dental and medical radiographic studies and modalities to render necessary diagnostic images and reports to the necessary third parties. In addition, the imaging center will be used to fabricate substructures for dental restorations as well as customized dental implant abutments, for distribution to third party referring dental labs. Moreover, this embodiment will also provide produces rapid prototype models that are utilized in pre-operative surgery planning, stent fabrication, and for patient education.

### BRIEF DESCRIPTION OF THE SEVERAL VIEWS OF THE DRAWING(S)

Not applicable

### DETAILED DESCRIPTION OF THE INVENTION

Referring now in more detail to a preferred embodiment of the present invention, the method and configuration for a Synergistic Medicodental outpatient imaging center comprises:

(a) A centralized center or outpatient health center for housing a plurality of medical radiographic modalities for diagnostic imaging purposes in combination with the dental radiographic modality Cone Beam CT volumetric scanner, also utilized for diagnostic purposes;

(b) performing a radiographic scan on the client's relevant anatomy with the relevant radiographic modality;

(c) generating a diagnostic radiographic image from the radiographic scan.

(d) storing the radiographic scans in an in medical imaging standard language technology that is capable of handling, storing, printing, and transmitting information.

(e) Maintaining images for storage, retrieval, distribution and presentation of images internally within the center and distribution to third party clients maintaining images in a picture archiving and communication systems (PACS);

(f) utilizing said PACS for the purpose of enabling the integration of scanners, servers, workstations, printers, and network hardware from multiple manufactures;

(g) when necessary converting said images into three dimensional reconstruction component present with both the medical and dental modalities;

(h) further including in the said center the capability for the fabrication of CAD/CAM milled dental substructures for dental custom implant abutments, and dental restorations made of zirconium and titanium for the purpose shipment to third party dental lab clients;

(I) further including the ability of the said center to have the said capability to utilize rapid model prototype fabrication for preoperative models and surgical stents for shipment to both dental as well as medical third party clients. The fabricated models would be based on information gathered from scans performed from both the dental and medical diagnostic radiographic modalities of the center. Fabrication of these models can occur either within the said center, or be assigned off site and returned to this primary center for final shipment to third party client; and

(j) further including in the said center or center utilization and capability to distribute condensed images at the point of completion of the scan directly to third party physician or dental client via electronic mail or e-mail and also the distribution of the primary radiologist report to clients by the same mode of distribution enabled by the customized practice management software within the center or outpatient health center.

Another preferred embodiment of the outpatient diagnostic imaging center, wherein the imaging center utilizes the following individual medical and dental modalities or any combinations of the medical and dental imaging modalities for the purpose of generating a diagnostic image, said medical and dental modalities including:
a Multislice (CT) Computed tomography Scanner, a Cone Beam Volumetric (CT) Computed tomography scanner, a (MRI) Magnetic Resonance Imaging Scanner (of various tesla strength), a Digital/Non Digital Mammography equipment, a Ultrasound equipment, a DEXA bone density equipment, a Digital X-Ray with or without fluoroscopy capabilities, a Positron emission tomography (PET) equipment, a Digital Dental Panorex equipment, a picture archiving and communication system (PACS), with Digital Imaging and a Communications in Medicine compatibility (DICOM) with a companion digital reading or workstation armamentarium.

Alternatively a preferred embodiment of the outpatient diagnostic imaging center wherein rapid prototype models are milled and fabricated based on a Computer Aided Design and/or animation developed off of the medical and dental modality scans, said milling and fabrication technologies for creating the rapid model prototype comprise:
selective sintering (SLS),
Fused Deposition Modeling (FDM),
Stereolithography (SLA),
Laminated Object Manufacturing (LOM),
Electron Beam Melting (EBM), and
3D printing (3DP)

What is claimed is:

1. A method of configuring a synergistic medicodental outpatient imaging center for the purpose of generating medical and dental diagnostic images, the method comprising acts of:
(a) locating an outpatient health center for housing a plurality of medical radiographic modalities for diagnostic imaging purposes in combination with a dental radiographic modality Cone Beam CT volumetric scanner, also utilized for diagnostic purposes;
(b) said medical and dental radiographic modalities comprise:
a Computed Tomography (CT) Multislice Scanner,
a dental radiographic modality Cone Beam CT volumetric
a Magnetic Resonance Imaging Scanner (MRI), Digital/Non Digital Mammography equipment,
an Ultrasound equipment,
a DEXA bone density equipment,
a Digital X-Ray with or without fluoroscopy capabilities,
a Positron emission tomography (PET) equipment, and
a Digital Dental Panorex equipment;
(c) arranging the medical and dental radiographic modalities in a specific combination network of equipment in said center;
(d) performing a radiographic scan on the client's relevant anatomy with the necessary radiographic modalities.

2. A method of configuring a diagnostic imaging center according to claim 1, wherein the radiographic scans are retained in a medical imaging standard language for handling, storing, printing, and transmitting information to an archiving system for storing, retrieving, distributing and presenting client images internally within the facility and to third party clients.

3. A method of configuring a diagnostic imaging center according to claim 2, wherein the archiving systems is used for the purpose of uploading and storing images for an extended period of time.

4. A method of configuring a diagnostic imaging center according to claim 2, comprising an image archiving system that would enable the integration of scanners, servers, workstations, printers, and network hardware from multiple manufactures.

5. A method of configuring a diagnostic imaging center according to claim 2, wherein said medical and dental radiographic modalities are all housed within one location.

6. A method of configuring a diagnostic imaging center according to claim 2, wherein the client can receive at least a medical diagnosis based on images generated from the necessary radiographic modalities.

7. A method of configuring a diagnostic imaging center according to claim 2, wherein the client can receive at least a dental diagnostic image based on images generated from the dental radiographic modalities.

8. A method of configuring a diagnostic imaging center according to claim 1, whereby a radiographic scan is taken and an image is generated, stored, and archived so that the staff radiologist can interpret the scan and prepare a preliminary or final report for the referring care provider.

9. A method of configuring a diagnostic imaging center according to claim 1, further including a LCD touch screen unit for the purpose of real time, instant, or virtual verification of patient's health insurance qualified benefits.

10. A method of configuring a diagnostic imaging center according to claim 9, wherein the client's insurance information is communicated to the LCD unit by means of a magnet strip.

11. A method of configuring a diagnostic imaging center according to claim 1, further including a Voice Recognition Dictation Technology integrated into the staff radiologist work stations.

12. A method of configuring a diagnostic imaging center according to claim 1, further including an Uninterruptible Power Supply, isolation transformers, and generators to provide uninterrupted, conditioned, clean power to the critical loads to back up the network of equipment.

13. A method of configuring a diagnostic imaging center according to claim 1, further comprising a CAD/CAM milling unit and software.

14. A method of configuring a diagnostic imaging center according to claim 1, further including a two way radio communication system as a standardized form of communication.

15. A method of configuring a diagnostic imaging center for the purpose of generating medical and dental diagnostic images, the method comprising of:
(a) an outpatient diagnostic imaging center for housing a plurality of medical radiographic modalities for diagnostic imaging purposes in combination with a dental radiographic modality Cone Beam CT volumetric scanner, also utilized for diagnostic purposes
(b) said medical and dental radiographic modalities comprise:
a Computed Tomography (CT) Multislice Scanner,
a dental radiographic modality Cone Beam CT volumetric
a Magnetic Resonance Imaging Scanner (MRI),
a Digital Mammography and Non Digital mammography equipment,
an Ultrasound equipment,
a DEXA bone density equipment,
a Digital X-Ray with or without fluoroscopy capabilities,
a Positron emission tomography (PET) equipment, and
a Digital Dental Panorex equipment;
(c) arranging the medical and dental radiographic modalities in a specific combination network of equipment in said center;
(d) the purpose of this previously mentioned configuration is for performing a radiographic scan on the client's relevant anatomy with the appropriate radiographic modality;
(e) said radiographic scan produces a diagnostic image;
(f) said diagnostic image is stored and transmitted in a medical imaging standard language.
(g) a CAD/CAM milling unit and software for creating 3D models from the diagnostic image
(h) a CAD/CAM milling unit and software further is for rapid prototype medical model fabrication.

16. A method of configuring a diagnostic imaging center according to claim 15, wherein the CAD/CAM milling unit is used to create dental substructures, dental abutments, and substructures for dental restorations, whereby the substructures for dental restorations are inclusive of crowns and bridges based the clients images generated from the radiographic modality.

17. A method of configuring a diagnostic imaging center according to claim 15, further including a Radiology Information System (RIS) and Hospital Information System (HIS) for managing patient information including scheduling, billing, shipping, records for CAD/CAM, and the records for milling dental crown and bridge.

18. A method of configuring a diagnostic imaging center according to claim 15, further including a rapid model prototype fabrication for preoperative models fabricated implant, and surgical stents for both medical and dental onsite patients and third party clients.

19. A method of configuring a diagnostic imaging center according to claim 15, whereby said milling and fabrication technologies for creating the rapid model prototype, based on a computer aided design, is created by any of the following means:
selective sintering (SLS),
Fused Deposition Modeling (FDM),
Stereolithography (SLA),
Laminated Object Manufacturing (LOM),
Electron Beam Melting (EBM), and
3D printing (3DP)

20. An imaging facility comprising:
(a) a plurality of dental and medical imaging modalities, to capture and generate diagnostic images;
(b) a Radiology Information System (RIS) and Hospital Information System (HIS) to monitor the work flow of the facilities and also address the practice management needs of the center;
(c) performing a radiographic scan on the client's relevant anatomy with the relevant radiographic modality;
(d) generating a diagnostic radiographic image from the radiographic scan;
(e) storing the radiographic scans in a in medical imaging standard language technology that is capable of handling, storing, printing, and transmitting information in the imaging standard language to an image archiving system;
(f) said image archiving system to store the generated images and make them available to the client physician, dentists, and nurses for diagnosis and treatment;
(g) a staff radiologist who can interpret the radiographic scan and prepare a preliminary or final report for the referring care provider;
(h) said archiving system for the purpose of enabling the integration of scanners, servers, workstations, printers, and network hardware from multiple manufactures;
(i) a 3D modeling component for converting said images into three dimensional reconstruction component present with both the medical and dental modalities;
(j) a laser printer to print film when requested by clients;
(k) a rapid prototype technologies utilized in the fabrication of medical and dental preoperative Models, fabricated based on information gathered from various scans based on various modalities and stored via PACS system;
(l) a CAD/CAM milling and fabrication unit for milling of dental zirconium and stainless steel or alloy substructures for Dental crown and bridge restorations; along with customized dental implant abutments fabricated from a CAD program;
(m) a two way radio communication system as a standardized form of communication;
(n) a LCD touch screen unit for the purpose of real time, instant, or virtual verification of patient's health insurance qualified benefits;
(o) a magnetic strip reader for said LCD touch screen unit;
(p) a Voice Recognition Dictation Technology integrated into the staff radiologist work stations;
(q) an Uninterruptible Power Supply, isolation transformers, and generators to provide uninterrupted, conditioned, clean power to the critical loads to back up the network of equipment.
